(19) United States
(12) Patent Application Publication
(10) Pub. No.: US 2014/0155736 A1
(43) Pub. Date: Jun. 5, 2014

(54) SYSTEM AND METHOD FOR AUTOMATED LANDMARKING

(71) Applicant: GENERAL ELECTRIC COMPANY, Schenectady, NY (US)

(72) Inventors: Vivek Prabhakar Vaidya, Bangalore (IN); Robert David Darrow, Niskayuna, NY (US); Xiaodong Tao, Beijing (CN)

(73) Assignee: GENERAL ELECTRIC COMPANY, Schenectady, NY (US)

(21) Appl. No.: 13/690,815

(22) Filed: Nov. 30, 2012

(30) Foreign Application Priority Data
Nov. 30, 2012 (IN) 4128/CHE/2011

(51) Int. Cl. A61B 6/04; A61B 5/055

(52) U.S. Cl. CPC A61B 6/04; A61B 5/0555
USPC 600/415; 600/407

(57) ABSTRACT

A method for automated landmarking comprising obtaining one or more localizer images of a patient, comparing the one or more localizer images with a reference image, computing a difference between the one or more localizer images and the reference image, determining a desired position of the patient based on the computed difference, and maneuvering the patient, a support platform, or both the patient and the support platform to the desired position for imaging an anatomical region of interest in the patient.

### BACKGROUND OF THE INVENTION

Embodiments of the present invention relate to imaging, and more particularly to automated landmarking for cardiac magnetic resonance imaging.

In the recent years, tomographic scanning techniques like magnetic resonance imaging (MRI) have been increasingly used in cardiology. Cardiac magnetic resonance imaging is rapidly becoming an important tool in the clinical management of cardiac patients. The ability of cardiac MRI to accurately characterize myocardial tissue has made cardiac MRI an invaluable tool for evaluating the anatomy and function of the heart, valves, major vessels, and surrounding structures, diagnose a variety of cardiovascular problems, detect and evaluate effects of coronary artery disease, plan and monitor treatment of a patient for cardiovascular problems. Cardiac MRI is also employed to evaluate the anatomy of the heart and blood vessels in children with congenital cardiovascular disease.

Typically, in cardiac MRI, volume scans of the thoracic region are acquired. However, in order to extract physiologically meaningful information from these thoracic scans, it is desirable to identify regions or features of interest. For example, cardiac MRI calls for accurate landmarking of the cardiac region/heart in a patient. Traditionally, use of currently available techniques entails localization of the feature of interest via manual operator intervention. Typically, this is done using external landmarks, such as the inferior edge of the sternum, as a reference. Although the location of the heart is related to the location of the sternum, this correlation is weak and will result in an incorrect field of view. Additionally, the process of landmarking is not always accurate, thereby calling for re-scans. Moreover, the determination of the landmark has a profound impact on other image analysis algorithms. Unfortunately, manual landmarking is a time-consuming process and requires human intervention.

Certain other currently available techniques entail landmarking of the region of interest in the patient using an in-built laser. In particular, the patient is disposed on a patient table and a coil is disposed on the patient. An in-built laser is then used to select a desired location. For example, the in-built laser is used to landmark the sternal notch in the patient. Subsequently, 3-plane localizer images with a relatively large field of view (FOV) and relatively low spatial resolution are acquired. Following this a series of images are obtained. This process is disadvantageously laborious and time-consuming. Additionally, any adjustment of the patient and/or a patient table involves manual movement. Consequently, time required for an MRI scan is increased, thereby resulting in a smaller number of patients being scanned in a given period of time.

### BRIEF SUMMARY OF THE INVENTION

According to an embodiment of the present invention, there is provided a method for automated landmarking The method comprises obtaining one or more localizer images of a patient, comparing the one or more localizer images with a reference image, computing a difference between the one or more localizer images and the reference image, determining a desired position of the patient based on the computed difference, and maneuvering the patient, a support platform, or both the patient and the support platform to the desired position for imaging an anatomical region of interest in the patient.

According to an embodiment of the present invention, there is provided a system for automated landmarking The system comprises a landmarking platform configured to obtain one or more localizer images of a patient, compare the one or more localizer images with a reference image, compute a difference between the one or more localizer images and the reference image, determine a desired position of the patient based on the computed difference, and maneuver the patient, a support platform, or both the patient and the support platform to the desired position.

According to an embodiment of the present invention, there is provided an imaging system. The system comprises an acquisition subsystem configured to obtain a plurality of image data sets corresponding to a region of interest in an object of interest, and a processing subsystem in operative association with the acquisition subsystem and comprising a landmarking platform, wherein the landmarking platform is configured to obtain one or more localizer images of the object of interest, compare the one or more localizer images with a reference image, compute a difference between the one or more localizer images and the reference image, determine a desired position of the object of interest based on the computed difference, and maneuver the object of interest, a support platform, or both the object of interest and the support platform to the desired position.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 is a flow chart depicting an exemplary method for automated landmarking/determination of an optimal position of a patient for cardiac MRI, in accordance with aspects of the present technique;

FIGS. 2A and 2B depict a flow chart embodying an exemplary method for automated landmarking/determination of an optimal position of a patient for cardiac MRI, in accordance with aspects of the present technique;

FIG. 3 is a flow chart depicting another exemplary method for automated landmarking/determination of an optimal position of a patient for cardiac MRI, in accordance with aspects of the present technique;

FIG. 4 is a diagrammatical illustration of a system for automated landmarking/determination of an optimal position of a patient for cardiac MRI, in accordance with aspects of the present technique; and

FIG. 5 is a diagrammatical illustration of a magnetic resonance imaging system for use in the system of FIG. 1.

### DETAILED DESCRIPTION OF THE INVENTION

As will be described in detail hereinafter, various systems and methods for automated landmarking for cardiac MRI are presented. The methods and systems described hereinafter provide a robust framework for the identification of an optimal imaging position of a patient for cardiac MRI with minimal effort. By employing the methods and systems described hereinafter, a framework that allows for automated landmarking yields a simpler, faster, and enhanced workflow.

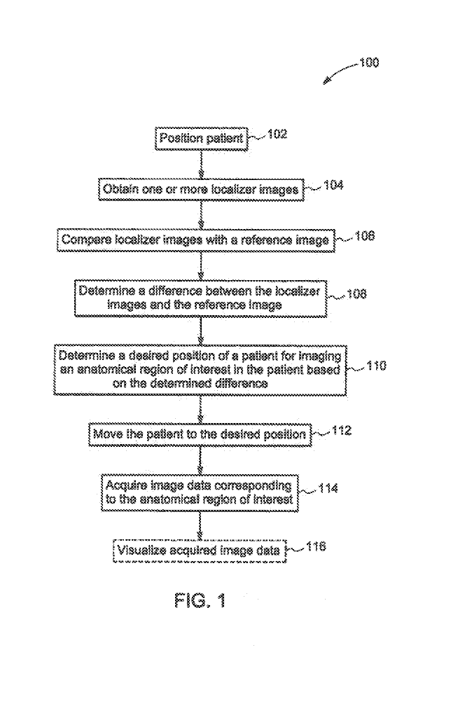

Referring now to FIG. 1, a flow chart 100 depicting an exemplary method for the automated landmarking of an anatomical region of interest is presented. As used herein, the term “landmarking” is used to refer to determination of an optimal position of a patient within a patient bore of an MRI system to aid in cardiac MRI, for example. Also, the terms landmarking and determining an optimal position or desired position of the patient may be used interchangeably. It may be noted that the method for automated landmarking of the anatomical region of interest in the patient is described with reference to imaging the cardiac region in the patient.

Also, the method starts when the patient is positioned 102 on a support platform, such as a patient table of the MRI system, for example. It may be noted that the terms support platform and patient table may be used interchangeably. Additionally, one or more imaging coils may be positioned on the patient. For example, for imaging the cardiac region in the patient, the imaging coils may be positioned on or around the thoracic/cardiac region of the patient.

Subsequently, one or more localizer images representative of the patient are acquired 104. These localizer images may be used in localizing the anatomical region of interest. In particular, the localizer images may be used to ensure that the anatomical region of interest, such as the cardiac region, for example, is located within the field of view of the one or more localizer images. The term field of view is used to refer to physical dimensions of acquisition, in one example. By way of example, images or image volumes representative of a thoracic and/or cardiac region of the patient may be acquired such that the images include the heart.

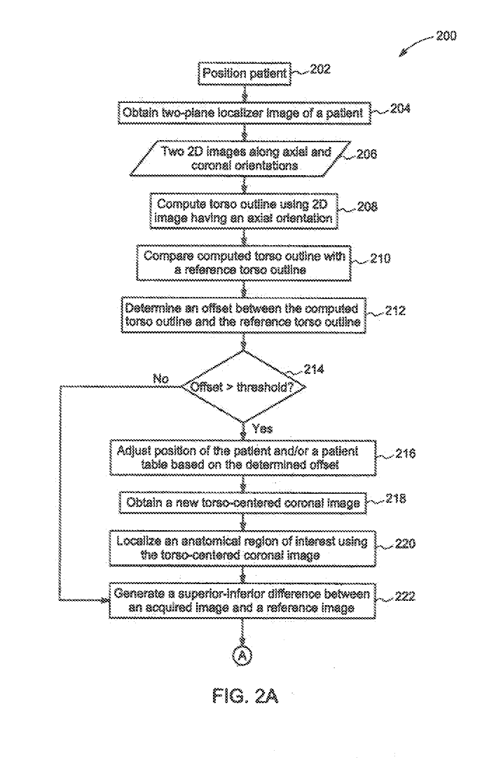

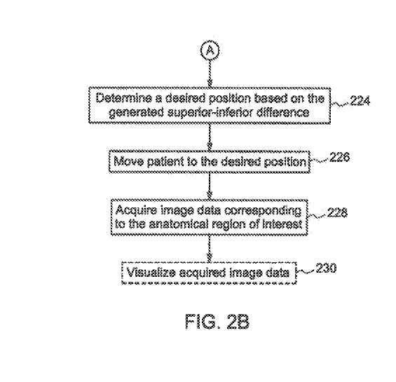

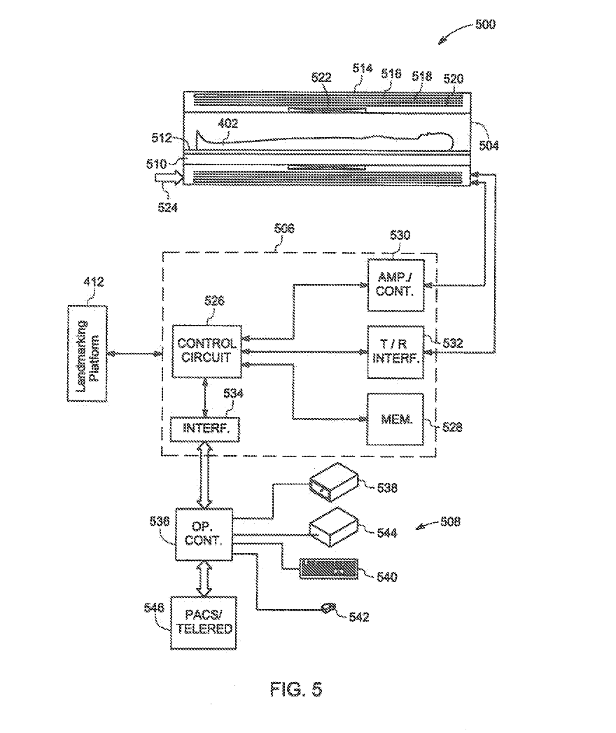

It may be noted that in certain embodiments, a magnetic resonance imaging (MRI) system (see FIG. 5) may be employed to acquire the one or more localizer images. In certain embodiments, other additional image acquisition devices, such as a charge coupled device (CCD) camera may be employed to acquire the one or more localizer images. Furthermore, in an embodiment, the CCD camera may be a part of the MRI system, while, in some embodiments, the CCD camera may be remote from the MRI system. The acquisition of the one or more localizer images will be described in greater detail with reference to FIGS. 2A-5.

Moreover, in an embodiment, the localizer images may be acquired employing a single modality imaging system. By way of example, the localizer images representative of the anatomical region of interest in the patient may be acquired using an MRI system. However, the one or more localizer images may also be acquired using a multi-modality imaging system. The multi-modality imaging system may include systems such as, but not limited to, positron emission tomography (PET)-MRI imaging systems, and the like. It may be noted that if the localizer images are acquired using a multi-modality imaging system, there may exist a lack of standardization between the multi-modal images. This lack of standardization may disadvantageously result in erroneous matching of the image volumes. In accordance with aspects of the present technique, if the one or more localizer images are acquired using a multi-modality imaging system, a feature space in each of the acquired localizer images may be standardized in order to match data in the multi-modal space.

Once the one or more localizer images are obtained, these localizer images may be compared with a reference image 106. The reference image may be representative of a previously acquired image corresponding to the region being imaged. By way of example, for imaging the cardiac region in the patient, the reference image may include a previously acquired image corresponding to the cardiac region. This comparison 106 ensures that the acquired localizer images are indeed representative of the desired anatomical region of interest.

Furthermore, a difference, if any, between the acquired localizer images and the reference image may be determined 108. The difference so generated may then be employed to determine a desired or optimal position of the patient and/or the patient table for imaging the desired anatomical region of interest in the patient 110. The desired or optimal position may generally be representative of a position of the patient and/or the patient table that allows the anatomical region of interest to be centered with respect to a portion of the MRI system, in one embodiment. In certain embodiments, the desired or optimal position may be representative of a position of the patient within the patient bore of the MRI system that allows imaging of enhanced quality of the anatomical region of interest.

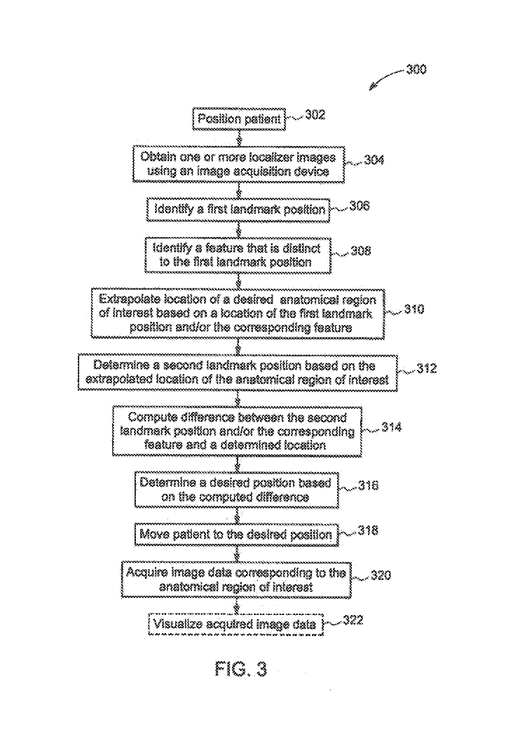

Subsequently, the patient and/or the patient table may be moved such that the anatomical region of the patient is positioned at the desired or optimal position based on the determined difference 112. Also, once the patient is located at the desired position, image data corresponding to the anatomical region of interest may be acquired 114. Moreover, the acquired image data may optionally be visualized on a display of the MRI imaging system 116. The method of FIG. 1 will be described in greater detail with reference to FIGS. 2A-3.

As noted hereinabove, the method of FIG. 1 may be performed using an MRI system or a combination of an image acquisition device such as a CCD camera and the MRI system. Turning now to FIGS. 2A and 2B, a flowchart 200 depicting an exemplary method for automated landmarking for cardiac MRI is depicted. The method starts when a patient is positioned 202 on a patient table of the MRI system, for example. Additionally, one or more imaging coils may be positioned on the patient. As previously noted, for imaging the cardiac region in the patient, the imaging coils may be positioned on or around the cardiac region of the patient. The patient may then be positioned within the patient bore of the MRI system.

In an embodiment, the patient is positioned at an optimal location within the patient bore to facilitate imaging of enhanced quality of the anatomical region of interest. In an embodiment, for cardiac MRI, the patient is positioned within the patient bore such that the cardiac region of the patient is centered with respect to the magnet in the MRI system.

To that end, one or more localizer images 206 may be obtained 204. As previously noted, the one or more localizer images 206 may be used to ensure that the anatomical region of interest lies within the field of view of the acquired localizer images. In an embodiment, the localizer images may include a two-plane localizer image 206. In particular, the two plane localizer image 206 may include two two-dimensional (2D) image slices. Specifically, one of the two 2D image slices may include a 2D image slice acquired along an axial direction, while the other 2D image slice may include a 2D image slice acquired along a coronal direction. These localizer images 206 may be acquired such that image data corresponding to a relatively large (large enough) field of view data in two planes is obtained. Specifically, image data may be acquired in the two planes to ensure that the anatomical region of interest is disposed within the two planes.

In accordance with aspects of the present invention, subsequent to the acquisition of the two plane localizer images 206, an outline of the torso of the patient may be computed using the axial 2D image slice 208. The computed torso outline may be employed to aid in localizing the anatomical region of interest. By way of example, for imaging the cardiac region, the lungs of the patient are localized.

The computed torso outline is then compared with a reference image 210. For example, the computed torso outline may be compared with a previously stored reference image that corresponds to the computed torso outline. In addition, the reference image may be scaled based on the computed torso outline 210. This scaling aids in normalizing across patient size and/or shape. For example, if the computed torso outline of the patient under observation is larger than the torso outline of the reference image, then the reference image may be scaled up to match the computed torso outline. However, if the computed torso outline of the patient under observation is smaller than the torso outline of the reference image, then the reference image may be scaled down to match the computed torso outline.

Subsequently, any offset between the computed torso outline and the torso outline corresponding to the reference image may be determined 212. As previously noted the reference outline may be scaled up or down to match the computed torso outline. This offset or difference aids in centering the torso of the patient within a region of acquisition. By way of example, the offset between the axial image and the reference image may include an offset in an anterior-posterior (AP) direction and/or in a left-right (LR) direction.

Once the offset is determined, a check may be carried out to verify if the offset is greater than or less than a determined threshold. The determined threshold may be representative of an acceptable offset between the computed torso outline and the reference torso outline. Accordingly, a check may be carried out to verify if the offset is greater than the determined threshold 214. If it is determined that the offset is greater than the determined threshold, then the position of the patient, the patient table, or both the patient and the patient table may be adjusted based on the determined offset 216. For example, the patient and/or the patient table may be translated along the AP direction to compensate for any offset in the AP direction. In a similar fashion, any offset in the LR direction may be compensated for by adjusting the position of the patient on the patient table. Adjusting the position of the patient and/or the patient table to compensate for any offset aids in centering the torso of the patient within the desired region of acquisition.

Subsequently, a new torso-centered coronal image may be acquired 218. In particular, a coronal image of the torso of the patient that is centered within the desired region of acquisition may be acquired. Additionally, the desired anatomical region of interest may be localized based on the torso-centered coronal image 220. By way of example, if the anatomical region of interest includes the heart, then the cardiac region may be localized using the torso-centered coronal image. In an embodiment, the torso-centered coronal image of the cardiac/thoracic region of the patient may be used to identify the lungs to aid in localizing the cardiac region. The lungs may be identified by determining large dark objects in the torso-centered coronal image, in an embodiment. Once the lungs are identified, the cardiac region may be localized. However, if it is verified that the offset is smaller than the determined threshold 214, then control may be passed on to generate a superior-inferior difference between an acquired image and a reference image 222.

In addition, once the anatomical region of interest is localized, a superior-inferior (SI) difference between the acquired image of the anatomical region of interest and a corresponding reference image may be generated 222. By way of example, if the desired anatomical region of interest includes the cardiac region, an image of the cardiac region may be acquired and compared with a reference image of the cardiac region to generate the SI difference image. It may be noted that in certain embodiments, the acquired image may be the torso-centered coronal image. However, in certain other embodiments, the acquired image may be other types of localizer scans. The SI difference image may then be employed to determine the optimal or desired position of the patient and/or the patient table 224. It may be noted that in certain embodiments, the desired or optimal position may generally be representative of a position of the patient and/or the patient table that allows the anatomical region of interest to be centered in the MRI system. In an embodiment, the desired position may include a position of the patient and/or the patient table such that the anatomical region of interest is centered in the middle of the MRI system. Also, in certain embodiments, the desired position may include a position of the patient and/or the patient table such that the anatomical region of interest is aligned or centered with respect to a position of the magnet of the MRI system. For example, while performing a cardiac MRI exam, the desired or optimal position may be representative of a position of the patient and/or patient table such that the heart of the patient is centered in the middle of the MRI system.

Subsequently, the patient and/or patient table may be maneuvered to the desired location 226. It may be noted that in certain embodiments, the patient table may be moved automatically to locate the anatomical region of interest of the patient at the desired position. However, in certain embodiments, any movement of the patient table to the desired position may entail manual maneuvering of the patient table. With the patient positioned at the optimal position, image data of the anatomical region of interest may be acquired 228. It may be noted that the acquisition of image data may be automated or manual. In certain embodiments, the image data corresponding to the anatomical region of interest may be acquired automatically. However, in certain embodiments, the acquisition of the image data may entail user intervention in the form of additional inputs. Positioning the patient at the optimal position as described hereinabove aids in acquiring image data corresponding to a relatively smaller region. The acquired image data may be processed to determine any disease state, track the progression of the disease state and/or monitor the efficacy of treatment on the disease state. Also, the acquired image data may optionally be visualized on a display of the imaging system 230.

As previously noted, in some embodiments, an image acquisition device, such as a CCD camera may be used to acquire the one or more localizer images. In accordance with aspects of the present invention, a method for the automated landmarking for cardiac MRI is presented. It may be noted that in certain embodiments, the CCD camera may be disposed remote from the MRI system. Alternatively, the CCD camera may be an integral part of the MRI system.

FIG. 3 is a flowchart 300 depicting an exemplary method for landmarking for cardiac MRI. In particular, an optimal position of the patient within the patient bore of the MRI system for imaging the cardiac region of the patient is presented. The method starts when a patient is positioned 302 on a patient table of the MRI system, for example. Moreover, one or more imaging coils may be positioned on the patient. As previously noted, for imaging the cardiac region in the patient, the imaging coils may be positioned on or around the cardiac region of the patient.

Furthermore, one or more localizer images may be obtained 304. In particular, an image acquisition device such as a CCD camera may be employed to acquire the localizer images. Moreover, in an embodiment, the localizer images may include video data corresponding to the patient. Also, a first landmark position on the imaging coil may be determined 306 using the localizer images acquired by the CCD camera. By way of example, in an embodiment, the CCD camera may be configured to identify a location of the imaging coil that is disposed on or about the patient.

Additionally, the acquired video and/or the first landmark position may be used to identify locations of one or more features of the patient 308. The one or more features may generally be representative of features in the vicinity of the cardiac region of the patient, for example. The face and/or arms of the patient may be identified 308, in an embodiment. To that end, in an example, a relationship between a location of the imaging coil and/or the first landmark position and various features such as the head, the arms or the legs on the body of the patient may be determined. Subsequently, a signature that is distinct to the identified features may be identified. The signature that is distinct to the face of the patient may include the eyes and/or the nose of the patient, for example. In a similar fashion, the signature that is distinct to the arms of the patient may include the shoulders.

Using the locations of the one or more features of interest and/or the corresponding signatures, a location of the desired anatomical region of interest may be extrapolated 310. For example, given the locations of the face and/or the eyes and the nose, and the location of the arms and/or the shoulders, the location of the cardiac region (desired anatomical region of interest) may be extrapolated. The extrapolated or estimated position of the anatomical region of interest may then be used to determine an appropriate landmark 312.

Moreover, a difference between the second landmark position and a determined location may be computed 314. In an example, the determined location may be representative of a position of the magnet in the MRI system. This difference may be used to determine an optimal or desired position of the patient and/or the patient table, as depicted by step 316. Subsequently, at step 318, the patient and/or the patient table may be moved to the desired position. It may be noted that in certain embodiments, the patient table may be moved automatically to the desired position. However, in certain other embodiments, the patient table may be manually moved to the desired position. With the patient positioned at the optimal or desired position, image data of the desired anatomical region of interest may be acquired 320. As previously noted with reference to FIGS. 2A and 2B, the image data may be automatically acquired. Alternatively, the acquisition of image data may entail user intervention. The acquired image data may be processed to determine any disease state. Also, the acquired image data may optionally be visualized on a display of the imaging system 322.

Positioning the patient at the desired position as described hereinabove aids in acquiring image data corresponding to a relatively smaller region. Additionally, positioning the patient at the optimal position aids in locating the anatomical region of interest closer to the center of the magnet in the MRI system. Consequently, the quality of image data acquired is enhanced as quality of the image data corresponding to the region of interest is substantially enhanced by positioning the patient as described hereinabove.

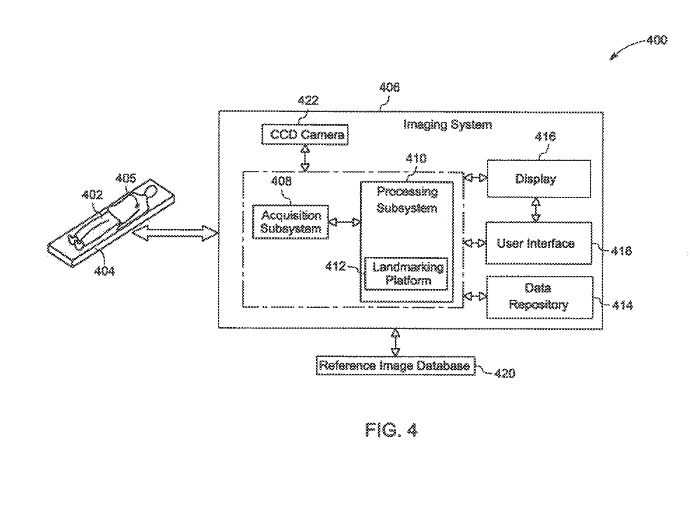

Turning now to FIG. 4, a block diagram of an exemplary system 400 for use in diagnostic imaging in accordance with aspects of the present invention is presented. The system 400 is configured to facilitate automated landmarking for cardiac magnetic resonance (MR) imaging. As previously noted, the system 400 aids in the automated determination of an optimal position of the patient within the patient bore of the imaging system for cardiac MR imaging. It may be noted that the system 400 may also be configured to assist in the automated and/or user-assisted landmarking for cardiac MR imaging of an object of interest, in certain embodiments. The object of interest may include a patient, in certain embodiments.

A patient 402 may be positioned on a support platform, such as a patient table 404. Additionally, one or more imaging coils 405 may be positioned on the patient 402. For example, for imaging the cardiac region in the patient 402, the imaging coils 405 may be positioned on or around the thoracic/cardiac region of the patient 402.

Moreover, the system 400 may also include a medical imaging system 406 that is configured to image the patient 402. It should be noted that although the exemplary embodiments illustrated hereinafter are described in the context of a medical imaging system, other imaging systems and applications such as industrial imaging systems and non-destructive evaluation and inspection systems, such as pipeline inspection systems, liquid reactor inspection systems, are also contemplated. In this case, it may be desirable to automatically determine a landmark for an object of interest.

Additionally, the exemplary embodiments illustrated and described hereinafter may find application in multi-modality imaging systems that employ MR imaging in conjunction with other imaging modalities, position-tracking systems or other sensor systems. For example, the multi-modality imaging system may include a positron emission tomography (PET) imaging system-MR imaging system. Furthermore, it should be noted that although the exemplary embodiments illustrated hereinafter are described in the context of a medical imaging system, such as an MRI system, use of other imaging systems, such as, but not limited to, a computed tomography (CT) imaging system, an X-ray imaging system, a PET imaging system, an ultrasound imaging system and other imaging systems is also contemplated in accordance with aspects of the present invention.

As noted hereinabove, in a presently contemplated configuration, the medical imaging system 406 may include a MR imaging system. The medical imaging system 406 may include an acquisition subsystem 408 and a processing subsystem 410, in an embodiment. Further, the acquisition subsystem 408 of the medical imaging system 406 is configured to acquire image data representative of one or more anatomical regions of interest in the patient 402, in an embodiment. In accordance with aspects of the present invention, the system 400 and the acquisition subsystem 408 in particular may be configured to acquire one or more localizer images of the patient 402. These localizer images may be used in localization of the anatomical region of interest, such as the cardiac region, for example.

Once the one or more localizer images are obtained, these localizer images may be processed by the processing subsystem 410. Particularly, the processing subsystem 410 may be configured to aid in the automated landmarking of the anatomical region of interest in the patient 402 for cardiac MRI. As previously noted, the automated landmarking entails determining an optimal or desired position of the patient 402 within the patient bore of the MRI system. To that end, in accordance with aspects of the present invention, the processing subsystem 410 includes a landmarking platform 412. The landmarking platform 412 may be configured to aid in the automated landmarking for cardiac MRI. To that end, the landmarking platform 412 may be configured to determine an optimal position of the patient 402 within the MRI system to facilitate imaging of enhanced quality.

In an example, the localizer images may include a two-plane localizer image such as the two-plane localizer image 206 of FIG. 2A that includes a 2D image slice acquired along an axial direction and another 2D image slice acquired along a coronal direction. The image data in the localizer images may be acquired in the two planes to ensure that the anatomical region of interest is disposed within the two planes.

In accordance with aspects of the present invention, subsequent to the acquisition of the two plane localizer images, the landmarking platform 412 may be configured to compute an outline of the torso of the patient 402 using the axial 2D image slice. The computed torso outline is used to localize the anatomical region of interest. By way of example, for imaging the cardiac region, it may be desirable to localize the lungs of the patient.

Furthermore, the landmarking platform 412 may also be configured to compare the computed torso outline with a reference image. The reference image that corresponds to the computed torso outline may be obtained from a reference image database 420, for example. In addition, the landmarking platform 412 may also be configured to scale the reference image based on the computed torso outline to normalize across patient size and/or shape. For example, if the computed torso outline of the patient under observation is smaller than the torso outline of the reference image, then the reference image may be scaled down to match the computed torso outline. Similarly, if the torso outline of the patient under observation is larger than the torso outline of the reference image, then the reference image may be scaled up to match the computed torso outline.

Moreover, the landmarking platform 412 may further be configured to determine any offset between the computed torso outline and the torso outline of the reference image. The landmarking platform 412 may be configured to use this offset or difference to center the torso of the patient within a region of acquisition. It may be noted that the offset between the axial image and the reference image may include an offset in the anterior-posterior (AP) direction and/or in the left-right (LR) direction.

Once the offset is determined, the landmarking platform 412 may be configured to verify if the offset is greater than or less than a determined threshold, where the determined threshold may be representative of an acceptable offset between the computed torso outline and the reference torso outline. If it is determined that the offset is greater than the determined threshold, then the landmarking platform 412 may be configured to adjust the position of the patient 402 and/or the position of the patient table 404 based on the offset. In an example, the patient 402 and/or the patient table 404 may be translated along the AP direction to compensate for any offset in the AP direction. Also, any offset in the LR direction may be compensated for by adjusting the position of the patient 402 on the patient table 404. It may be noted that in certain embodiments, the landmarking platform 412 may be configured to automatically adjust the position of the patient table 404. However, in certain other embodiments, the position of the patient 402 and/or the patient table 404 may be manually maneuvered. Compensating for any offset by adjusting the position of the patient 402 and/or the patient table 404, the torso of the patient 402 may be centered within a desired region of acquisition, thereby enhancing the quality of the acquired image data.

Once the offset is compensated for, the landmarking platform 412 may be configured to aid in acquiring a new torso-centered coronal image. Particularly, a coronal image of the torso of the patient that is centered within the desired region of acquisition may be acquired. In an example, the landmarking platform 412 may control acquisition of the torso-centered coronal image using the acquisition subsystem 408.

Additionally, the landmarking platform 412 may also be configured to localize the anatomical region of interest using the torso-centered coronal image. As previously noted with reference to FIGS. 2A and 2B, the landmarking platform 412 may use the torso-centered coronal image of the cardiac/thoracic region of the patient to identify the lungs, thereby localizing the cardiac region.

Subsequent to localizing the anatomical region of interest, the landmarking platform 412 may be configured to generate a superior-inferior (SI) difference between the acquired image of the anatomical region of interest and a corresponding reference image. As previously noted, acquired image may include the torso-centered coronal image or other localizer scan images. Furthermore, the landmarking platform 412 is configured to use the SI difference image to determine a desired position of the patient 402 and/or the patient table 404.

As previously noted, in certain embodiments, a CCD camera 422 may be employed to acquire the localizer images. In an example, the localizer images may include video data corresponding to the patient 402. In this example, the landmarking platform 412 may be configured to determine a landmark position on the imaging coil 405 using the localizer images acquired by the CCD camera.

The landmarking platform 412 may also be configured to identify locations of one or more features of the patient 402 within a vicinity of the anatomical region of interest using the acquired video data. In one example, the face and/or arms of the patient 402 may be identified. Also, a signature that is distinct to the identified features may be determined. Using the locations of the one or more features of interest and the corresponding signatures, the landmarking platform 412 may be configured to extrapolate a location of the anatomical region of interest. By way of example, given the locations of the face and/or the eyes and the nose, and the arms and/or shoulders, the location of the cardiac region (desired anatomical region of interest) may be extrapolated. The landmarking platform 412 may be configured to use the extrapolated location to determine a landmark position.

Moreover, the landmarking platform 412 may also be configured to compute a difference between the landmark position(s) and a determined location. In one example, the determined location may be representative of a position of the magnet in the MRI system. In certain other embodiments, the determined location may be representative of the middle of the MRI system. This difference may be used to determine an optimal or desired position of the patient and/or the patient table.

The landmarking platform 412 may be configured to maneuver the patient 402 and/or patient table 404 to the optimal or desired position. In particular, the patient 402 and/or the patient table 404 may be maneuvered such that the anatomical region of interest of the patient 402 is located at the desired position. In certain embodiments, the landmarking platform 412 may be configured to automatically move the patient table 404 to the desired position. However, in certain other embodiments, the patient table 404 may be manually moved to the desired position.

With the patient 402 and more particularly the anatomical region of interest positioned at the optimal desired position, the landmarking platform 412 may be configured to facilitate acquisition of image data corresponding to the anatomical region of interest. It may be noted that the acquisition of image data may be automated or manual. In certain embodiments, the image data corresponding to the anatomical region of interest may be acquired automatically. However, in certain other embodiments, the acquisition of the image data may entail user intervention in the form of additional inputs. The acquired image data may be processed by the landmarking platform 412 or the processing subsystem 410 to determine any disease state, track the progression of the disease state and/or monitor the efficacy of treatment on the disease state.

The image data acquired and/or processed by the medical imaging system 406 may be employed to aid a clinician in identifying disease states, assessing need for treatment, determining suitable treatment options, tracking the progression of the disease, and/or monitoring the effect of treatment on the disease states. In certain embodiments, the processing subsystem 410 may be further coupled to a storage system, such as the data repository 414, where the data repository 414 is configured to store the acquired image data.

Further, as illustrated in FIG. 4, the medical imaging system 406 may include a display 416 and a user interface 418. In certain embodiments, such as in a touch screen, the display 416 and the user interface 418 may overlap. Also, in some embodiments, the display 416 and the user interface 418 may include a common area. Additionally, the acquired image data may be visualized on the display 416.

In addition, the user interface 418 of the medical imaging system 406 may include a human interface device (not shown) configured to aid the clinician in manipulating image data displayed on the display 416. The human interface device may include a mouse-type device, a trackball, a joystick, a stylus, or a touch screen configured to facilitate the clinician to identify the one or more regions of interest requiring therapy. However, as will be appreciated, other human interface devices, such as, but not limited to, a touch screen, may also be employed. Furthermore, in accordance with aspects of the present technique, the user interface 418 may be configured to aid the clinician in navigating through the images acquired by the medical imaging system 406. Additionally, the user interface 418 may also be configured to aid in manipulating and/or organizing the acquired image data displayed on the display 416.

As previously noted with reference to FIG. 4, the medical imaging system 106 may include an MR imaging system. FIG. 5 depicts a block diagram of an embodiment of an MRI imaging system 500. The MRI system 500 is illustrated diagrammatically as including a scanner 504, scanner control circuitry 506, and system control circuitry 508. While the MRI system 500 may include any suitable MRI scanner or detector, in the illustrated embodiment the system 500 includes a full body scanner including a patient bore 510 into which a cradle 512 may be positioned to place an object of interest in a desired position for scanning It may be noted that in an embodiment, the object of interest may include a patient, such as the patient 402 of FIG. 4. In one embodiment, the cradle may be representative of the patient table 404 of FIG. 4. The scanner 504 may be of any suitable field strength, including scanners varying from 0.5 Tesla to 3 Tesla and beyond. As used herein, the term patient is used to refer to a human person or animal that is the subject of the imaging application.

Additionally, the scanner 504 may include a series of associated coils for producing controlled magnetic fields, for generating radio-frequency (RF) excitation pulses, and for detecting emissions from gyromagnetic material within the patient 402 in response to such pulses. In the diagrammatical view of FIG. 5, a primary magnet coil 514 may be provided for generating a primary magnetic field generally aligned with the patient bore 510. A series of gradient coils 516, 518 and 520 may be grouped in a coil assembly for generating controlled magnetic field gradients during examination sequences as will be described in greater detail hereinafter. A RF coil 522 may be provided for generating radio frequency pulses for exciting the gyromagnetic material. In the embodiment illustrated in FIG. 5, the coil 522 also serves as a receiving coil. Thus, the RF coil 522 may be coupled with driving and receiving circuitry in passive and active modes for receiving emissions from the gyromagnetic material and for applying RF excitation pulses, respectively. Alternatively, various configurations of receiving coils may be provided separate from the RF coil 522. Such coils may include structures specifically adapted for target anatomies, such as head coil assemblies, and so forth. Moreover, receiving coils may be provided in any suitable physical configuration, including phased array coils, and so forth.

In a presently contemplated configuration, the gradient coils 516, 518 and 520 may have different physical configurations adapted to their function in the imaging system 500. As will be appreciated by those skilled in the art, the coils include conductive wires, bars or plates that are wound or cut to form a coil structure that generates a gradient field upon application of control pulses as described below. The placement of the coils within the gradient coil assembly may be done in several different orders. In an embodiment, a Z-axis coil may be positioned at an innermost location, and may be formed generally as a solenoid-like structure that has relatively little impact on the RF magnetic field. Thus, in the illustrated embodiment, the gradient coil 520 is the Z-axis solenoid coil, while coils 516 and 518 are Y-axis and X-axis coils respectively.

The coils of the scanner 504 may be controlled by external circuitry to generate desired fields and pulses, and to read signals from the gyromagnetic material in a controlled manner. As will be appreciated by those skilled in the art, when the material, typically bound in tissues of the patient 402, is subjected to the primary field, individual magnetic moments of the paramagnetic nuclei in the tissue partially align with the field. While a net magnetic moment is produced in the direction of the polarizing field, the randomly oriented components of the moment in a perpendicular plane generally cancel one another. During an examination sequence, an RF frequency pulse is generated at or near the Larmor frequency of the material of interest, resulting in rotation of the net aligned moment to produce a net transverse magnetic moment. This transverse magnetic moment precesses around the main magnetic field direction, emitting RF signals that are detected by the scanner 504 and processed for reconstruction of the desired image.

The gradient coils 516, 518 and 520 may be configured to serve to generate precisely controlled magnetic fields, the strength of which vary over a predefined field of view, typically with positive and negative polarity. When each coil is energized with known electric current, the resulting magnetic field gradient is superimposed over the primary field and produces a desirably linear variation in the Z-axis component of the magnetic field strength across the field of view. The field varies linearly in one direction, but is homogenous in the other two. The three coils have mutually orthogonal axes for the direction of their variation, enabling a linear field gradient to be imposed in an arbitrary direction with an appropriate combination of the three gradient coils.

The pulsed gradient fields perform various functions integral to the imaging process. Some of these functions are slice selection, frequency encoding and phase encoding. These functions may be applied along the X-axis, Y-axis and Z-axis of the original coordinate system or along other axes determined by combinations of pulsed currents applied to the individual field coils.

The slice select gradient determines a slab of tissue or anatomy to be imaged in the patient 402. The slice select gradient field may be applied simultaneously with a frequency selective RF pulse to excite a known volume of spins within a desired slice that precess at the same frequency. The slice thickness is determined by the bandwidth of the RF pulse and the gradient strength across the field of view.

The frequency encoding gradient is also known as the readout gradient, and is usually applied in a direction perpendicular to the slice select gradient. In general, the frequency encoding gradient is applied before and during the formation of the magnetic resonance (MR) echo signal resulting from the RF excitation. Spins of the gyromagnetic material under the influence of this gradient are frequency encoded according to their spatial position along the gradient field. By Fourier transformation, acquired signals may be analyzed to identify their location in the selected slice by virtue of the frequency encoding.

Finally, the phase encode gradient is generally applied before the readout gradient and after the slice select gradient. Localization of spins in the gyromagnetic material in the phase encode direction may be accomplished by sequentially inducing variations in phase of the precessing protons of the material using slightly different gradient amplitudes that are sequentially applied during the data acquisition sequence. The phase encode gradient permits phase differences to be created among the spins of the material in accordance with their position in the phase encode direction.

As will be appreciated by those skilled in the art, a great number of variations may be devised for pulse sequences employing the exemplary gradient pulse functions described hereinabove as well as other gradient pulse functions not explicitly described here. Moreover, adaptations in the pulse sequences may be made to appropriately orient both the selected slice and the frequency and phase encoding to excite the desired material and to acquire resulting MR signals for processing.

The coils of the scanner 504 are controlled by the scanner control circuitry 506 to generate the desired magnetic field and RF pulses. In the diagrammatical view of FIG. 5, the scanner control circuitry 506 thus includes a control circuit 526 for commanding the pulse sequences employed during the examinations, and for processing received signals. The control circuit 526 may include any suitable programmable logic device, such as a CPU or digital signal processor of a general purpose or application-specific computer. Also, the control circuit 526 may further include memory circuitry 528, such as volatile and non-volatile memory devices for storing physical and logical axis configuration parameters, examination pulse sequence descriptions, acquired image data, programming routines, and so forth, used during the examination sequences implemented by the scanner.

Interface between the control circuit 526 and the coils of the scanner 504 is managed by amplification and control circuitry 530 and by transmission and receive interface circuitry 532. The amplification and control circuitry 530 includes amplifiers for each gradient field coil to supply drive current to the field coils in response to control signals from the control circuit 526. The transmit/receive (T/R) circuitry 532 includes additional amplification circuitry for driving the RF coil 522. Moreover, where the RF coil 522 serves both to emit the RF excitation pulses and to receive MR signals, the T/R circuitry 532 may typically include a switching device for toggling the RF coil between active or transmitting mode, and passive or receiving mode. A power supply, denoted generally by reference numeral 524 in FIG. 5, is provided for energizing the primary magnet 514. Finally, the scanner control circuitry 506 may include interface components 534 for exchanging configuration and image data with the system control circuitry 508. It should be noted that, while in the present description reference is made to a horizontal cylindrical bore imaging system employing a superconducting primary field magnet assembly, the present technique may be applied to various other configurations, such as scanners employing vertical fields generated by superconducting magnets, permanent magnets, electromagnets or combinations of these means.

The system control circuitry 508 may include a wide range of devices for facilitating interface between an operator or radiologist and the scanner 504 via the scanner control circuitry 506. In the illustrated embodiment, for example, an operator controller 536 is provided in the form of a computer workstation employing a general purpose or application-specific computer. The workstation also typically includes memory circuitry for storing examination pulse sequence descriptions, examination protocols, user and patient data, image data, both raw and processed, and so forth. Further, the workstation may further include various interface and peripheral drivers for receiving and exchanging data with local and remote devices. In the illustrated embodiment, such devices include a conventional computer keyboard 540 and an alternative input device such as a mouse 542. A printer 544 may be provided for generating hard copy output of documents and images reconstructed from the acquired data. Moreover, a computer monitor 538 may be provided for facilitating operator interface. In addition, the system 500 may include various local and remote image access and examination control devices, represented generally by reference numeral 546 in FIG. 5. Such devices may include picture archiving and communication systems, teleradiology systems, and the like. Additionally, the imaging system 500 includes the exemplary landmarking platform 412 of FIG. 1. Although the present example depicts the landmarking platform 412 as being coupled to the imaging system 500, it may be noted that in certain embodiments, the landmarking platform 412 may be an integral part of the imaging system 500.

In accordance with an aspect of the present invention, a method for automated landmarking is presented. The method includes obtaining one or more localizer images of a patient. Furthermore, the method includes comparing the one or more localizer images with a reference image. In addition, the method includes computing a difference between the one or more localizer images and the reference image. The method also includes determining a desired position of the patient based on the computed difference. Moreover, the method includes maneuvering the patient, a support platform, or both the patient and the support platform to the desired position for imaging an anatomical region of interest in the patient.

In accordance with an aspect of the present invention, a system for automated landmarking is presented. The system includes a landmarking platform, configured to obtain one or more localizer images of a patient, compare the one or more localizer images with a reference image, compute a difference between the one or more localizer images and the reference image, determine a desired position of the patient based on the computed difference, and maneuver the patient, a support platform, or both the patient and the support platform to the desired position.

In accordance with an aspect of the present invention, an imaging system is presented. The system includes an acquisition subsystem configured to obtain a plurality of image data sets corresponding to a region of interest in an object of interest. Additionally, the system includes a processing subsystem in operative association with the acquisition subsystem and including a landmarking platform, wherein the landmarking platform is configured to obtain one or more localizer images of the object of interest, compare the one or more localizer images with a reference image, compute a difference between the one or more localizer images and the reference image, determine a desired position of the object of interest based on the computed difference, and maneuver the object of interest, a support platform, or both the object of interest and the support platform to the desired position.

The various systems and methods for automated landmarking for cardiac MR imaging described hereinabove provides a framework for robust determination of a landmark for imaging a desired anatomical region of interest, such as the heart. The framework described hereinabove allows for automated determination of the landmark by leveraging a two plane localizer and/or acquired video data. Additionally, movement of the patient table may be automated, thereby yielding a simpler, faster, and enhanced imaging workflow. Furthermore, aligning the axial and coronal localizer images with a reference image aids in determining a physical distance between current location and the desired location associated with scan type. Also, the patient table is adjusted based on the generated difference, thereby enhancing the quality of imaging. Moreover, the automated technique advantageously aids in reducing the time required for an MRI scan, thereby enabling more patients to be scanned during a given time period. Additionally, need for human intervention is dramatically reduced

While only certain embodiments of the present invention have been illustrated and described herein, many modifications and changes will occur to those skilled in the art. It is, therefore, to be understood that the appended claims are intended to cover all such modifications and changes as fall within the true spirit of the present invention.

What is claimed is:

1. A method for automated landmarking, the method comprising:
obtaining one or more localizer images of a patient;
comparing the one or more localizer images with a reference image;
computing a difference between the one or more localizer images and the reference image;
determining a desired position of the patient based on the computed difference; and
maneuvering the patient, a support platform, or both the patient and the support platform to the desired position for imaging an anatomical region of interest in the patient.

2. The method of claim 1, further comprising:
disposing the patient on or about the support platform; and
positioning at least one imaging coil on or about the anatomical region of interest in the patient.

3. The method of claim 2, wherein obtaining one or more localizer images of a patient comprises acquiring one or more images in an axial orientation, a coronal orientation, or both the axial and the coronal orientations.

4. The method of claim 3, further comprising computing an outline of a torso of the patient using the image acquired in the axial orientation.

5. The method of claim 4, further comprising:
comparing the computed outline of the torso with a reference image; and
determining an offset between the computed outline of the torso and the reference image.

6. The method of claim 5, further comprising:
adjusting the position of the patient, the support platform, or both the patient and the support platform based on the determined offset; and
obtaining a torso-centered image acquired in the coronal orientation.

7. The method of claim 6, further comprising localizing the anatomical region of interest using the torso-centered image acquired in the coronal orientation.

8. The method of claim 7, further comprising computing a difference between the torso-centered image acquired in the coronal orientation and the reference image.

9. The method of claim 8, further comprising determining a desired position of the patient based on the computed difference.

10. The method of claim 9, further comprising acquiring image data corresponding to the anatomical region of interest with the patient positioned at the desired position.

11. The method of claim 10, further comprising visualizing the acquired image data on a display.

12. The method of claim 2, wherein obtaining the one or more localizer images comprises acquiring the one or more localizer images using an image acquisition device.

13. The method of claim 12, further comprising:
determining a first landmark position using the one or more localizer images; and
identifying a feature of interest corresponding to the first landmark position.

14. The method of claim 13, further comprising:
extrapolating a location of the anatomical region of interest based on a location of the first landmark position and a location of the feature of interest; and
determining a second landmark position based on the extrapolated location of the anatomical region of interest.

15. The method of claim 14, further comprising:
computing an offset between the second landmark position and a determined location; and
determining a desired position of the patient based on the computed offset.

16. The method of claim 15, further comprising acquiring image data corresponding to the anatomical region of interest with the patient positioned at the desired position.

17. A system for automated landmarking, the system comprising:
a landmarking platform configured to:
obtain one or more localizer images of a patient;
compare the one or more localizer images with a reference image;
compute a difference between the one or more localizer images and the reference image;
determine a desired position of the patient based on the computed difference; and
maneuver the patient, a support platform, or both the patient and the support platform to the desired position.

18. An imaging system, comprising:
an acquisition subsystem configured to obtain a plurality of image data sets corresponding to a region of interest in an object of interest; and
a processing subsystem in operative association with the acquisition subsystem and comprising a landmarking platform, wherein the landmarking platform is configured to:
obtain one or more localizer images of the object of interest;
compare the one or more localizer images with a reference image;
compute a difference between the one or more localizer images and the reference image;
determine a desired position of the object of interest based on the computed difference; and
maneuver the object of interest, a support platform, or both the object of interest and the support platform to the desired position.

19. The system of claim 18, wherein the system comprises a magnetic resonance imaging system, a computed tomography imaging system, a positron emission tomography imaging system, or combinations thereof.
